(19) United States
(12) Patent Application Publication
(10) Pub. No.: US 2015/0119613 A1
(43) Pub. Date: Apr. 30, 2015

(54) PYROLYSIS GASOLINE TREATMENT PROCESS

(71) Applicant: UOP LLC, Des Plaines, IL (US)

(72) Inventors: Robert J. Schmidt, Barrington, IL (US); Charles P. Luebke, Mount Prospect, IL (US); Rose M. Janulis, Oak Brook, IL (US)

(73) Assignee: UOP LLC, Des Plaines, IL (US)

(21) Appl. No.: 14/063,480

(22) Filed: Oct. 25, 2013

(51) Int. Cl. C07C 7/163

(52) U.S. Cl. CPC C07C 7/163
USPC 585/251

(57) ABSTRACT

A process for treating pyrolysis gasoline that includes introducing a pyrolysis gasoline stream into a first stage reactor and performing a fractionation process on the pyrolysis gasoline stream after being routed through the first stage reactor. After performing the fractionation process, splitting the resultant stream is split into a first stream and a second stream. Next, the first stream is routed to a first portion of a second stage reactor and the second stream is routed to a second portion of the second stage reactor. Preferably, the first stage reactor is a di-olefin reactor, and the second stage reactor is a hydrotreater reactor.

The present invention relates generally to processes for treating pyrolysis gasoline, and more specifically to processes for treating pyrolysis gasoline to remove dienes and olefins prior to downstream processing to remove benzene, toluene and xylene isomers (commonly referred to as BTX processing).

### BACKGROUND OF THE INVENTION

The treatment of pyrolysis gasoline to remove dienes and olefins prior to downstream BTX processing for high value para-xylene (PX) remains a challenge. Currently, the process requires two steps and the high heat of reaction needed for these steps require high effluent recycle rates to maintain the resulting temperature rise at an acceptable delta temperature performance. The key steps include: (1) a first stage to saturate di-olefins; and (2) a second stage to hydrotreat the remaining olefins and aromatics to remove sulfur and nitrogen species down to a level of less than 0.5 ppm to make the net product stream acceptable for further processing in a downstream aromatics complex for high value PX production. The current technology is limited in that heat control in the first and second stages requires high selectivity catalysts to be used in the lead stage, followed by careful heat management in the second stage to reduce recycle rates to minimize utilities consumption and capital costs.

### BRIEF SUMMARY OF THE INVENTION

Briefly, in certain embodiments, the present process is a process for treating pyrolysis gasoline that includes introducing a pyrolysis gasoline stream into a first stage reactor and performing a fractionation process on the pyrolysis gasoline stream after being routed through the first stage reactor. After performing the fractionation process, the resultant stream is split into a first stream and a second stream. Next, the first stream is routed to a first portion of a second stage reactor and the second stream is routed to a second portion of the second stage reactor. Preferably, the first stage reactor is a di-olefin reactor, and the second stage reactor is a hydrotreater reactor.

Alternatively, in certain embodiments, the present process relates to a process for treating pyrolysis gasoline that includes routing a pyrolysis gasoline stream to a di-olefin reactor and then routing a first recycle liquid stream to the di-olefin reactor. The process of these embodiments also includes performing a fractionation process on the pyrolysis gasoline stream after being routed through the di-olefin reactor. Next, after performing the fractionation process, the resultant stream is split into a first stream and a second stream. The first stream is routed to a first portion of a hydrotreater reactor, and the second stream is routed to a second portion of the hydrotreater reactor. Additionally, preferably, a second recycle liquid stream is routed to the second portion of the hydrotreater reactor.

As an additional alternative, embodiments of the present process also relate to a process for treating pyrolysis gasoline that includes routing a pyrolysis gasoline stream containing a full range of C5 to C10 hydrocarbons to a first stage reactor and separating the C5 hydrocarbons and the C10 hydrocarbons from the pyrolysis gasoline stream after being routed through the first stage reactor. After separating, a liquid effluent stream containing C6 to C9 hydrocarbons is routed to a second stage reactor.

These and other embodiments are described in the following Detailed Description of the Invention.

### BRIEF DESCRIPTION OF THE DRAWING

A preferred embodiment of the present invention is described herein with reference to the drawing wherein:

FIG. 1 is an example of an embodiment of the present process for treating pyrolysis gasoline.

### DETAILED DESCRIPTION OF THE INVENTION

In certain embodiments, the present invention relates to a process for treating pyrolysis gasoline that utilizes a high selectivity di-olefin saturation catalyst consisting of a shell impregnated palladium (Pd) system or a Pd layered sphere system in the lead stage of a two stage reactor system, such that once through hydrogen (H₂) can be processed without excessive heat generation due to secondary saturation of olefins or aromatics. This first stage of the process is followed by a second stage where a high selectivity catalyst is used to selectively saturate the remaining olefins, and to hydrotreat the sulfur and nitrogen species without aromatics saturation. In the second stage, the catalyst could consist of a combination of a Ni—Mo catalyst and a Co—Mo catalyst in a system in which the ratio of Ni—Mo to Co—Mo of between 20% to 80% Ni—Mo catalyst and between 30% to 70% Co—Mo catalyst.

Further, as described more fully below, a split feed reactor can be used in the second stage. Under such a configuration, a gas phase only recycle stream is required to manage the heat, and the need for a liquid phase recycle stream is either eliminated, or, if desired, it could possibly be included as a back-up only for added process flexibility.

By using a high selectivity catalyst in the lead stage, the fractionation process can be performed between the first and second stages, enabling the achievement of a high yield with minimum recycle. Examples of the high selectivity catalyst include an egg shell type catalyst, ECS (engineered catalyst support or layer sphere system), and conventional uniformly impregnated Pd catalysts, such as PF-4. The egg shell type catalyst provides somewhat better selectivity than the ECS catalyst, and both the egg shell type catalyst and the ECS type catalyst provide better performance than the conventional uniformly impregnated Pd catalyst, PF-4.

Further, the use of a split feed reactor in the second stage allows the additional heat sink required in a liquid recycle to be eliminated, resulting in further capital and utilities cost reduction.

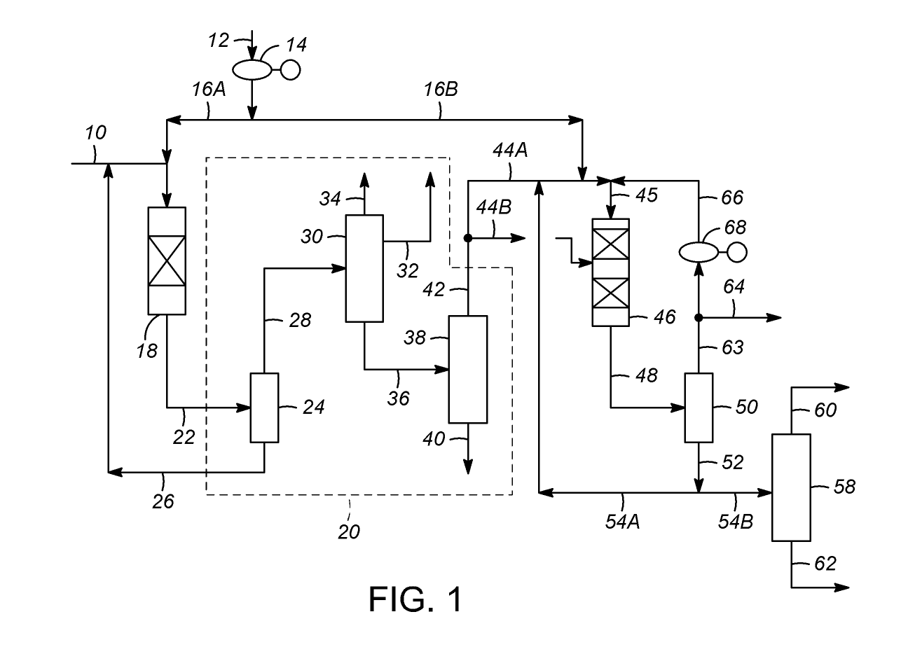

An example of an embodiment of the present process will now be described. More specifically, FIG. 1 is a process flow diagram that shows one example of a process for treating pyrolysis gasoline. Of course, other embodiments are also contemplated, as well as modifications to the FIG. 1 embodiment. Also, FIG. 1 is merely a schematic of the process flow, and therefore various features (such as processors, controllers, valves, sensors, etc.) are not shown. However, such additional features are known to those of ordinary skill in the art, and therefore are not necessary for an understanding or implementation of the present process.

The feed stream 10 of FIG. 1 is a pyrolysis gasoline stream that preferably contains a full range of C5 to C10 hydrocarbons. Preferably, the pyrolysis gasoline stream 10 is in the liquid phase, and is at a temperature within the range of 40° C. to 60° C. in the inlet of the first stage catalyst bed, and a pressure within the range of 350 to 850 psig, but at a minimum, a pressure high enough to maintain substantially all of the hydrocarbons in the liquid phase. In this embodiment, a make-up hydrogen stream 12 is introduced into a make-up hydrogen compressor 14 prior to being split into a first make-up hydrogen stream 16A and a second make-up hydrogen stream 16B. The make-up hydrogen streams 16A and 16B are controlled according to any desired method to provide the necessary make-up hydrogen to the associated stream, such as the pyrolysis gasoline stream 10. Although the make-up hydrogen streams 16A and 16B are in the vapor phase, they are being combined in such low percentages (for example about 2-3%) with the liquid phase streams (such as the pyrolysis gasoline stream 10 or stream 44A), that the gas phase hydrogen quickly dissolves, and the resulting combined stream remains in liquid phase.

After receiving make-up hydrogen from the make-up hydrogen stream 16A, if necessary, the pyrolysis gasoline stream 10 is directed to a first stage reactor 18, which in this embodiment is a di-olefin reactor that is used for removing di-olefins from the pyrolysis gasoline with a catalyst. Preferably, the catalyst used in the di-olefin reactor 18 is a high selectivity di-olefin saturation catalyst. For example, a high selectivity di-olefin saturation catalyst consisting of a shell impregnated palladium (Pd) system or a Pd layered sphere could be used. Alternatively, the catalyst could include engineered catalyst support (ECS). Sufficient performance could also be obtained with a conventional PF-4 catalyst, which is a spherical R-9 catalyst with 0.4% Pd, 0.5% Li that has been reduced and cold sulfided, although catalysts with an eggshell Pd profile are preferred for certain embodiments.

The first stage reactor 18 may be of any desired type, but one example of a specific embodiment of a two bed reactor that can be used in the present process is disclosed in Application Serial No. ______ [GBC Docket No. 5066.115488; UOP No. H0041208-8200], which is assigned to the same Assignee as the present application, and which is hereby incorporated by reference it its entirety into the present application.

After the pyrolysis gasoline has been routed through the first stage reactor 18, a fractionation process can be performed upon the pyrolysis gasoline stream. Dashed box 20 of FIG. 1 contains one example of a fractionation process that can be used to separate the C5 and the C9+ hydrocarbons from the stream, but of course other configurations of components and processes for fractionation are also contemplated. In the fractionation process 20, stream 22 is routed to a first stage surge drum 24. A resultant liquid stream 26 from the surge drum 24 is routed as a recycle stream that is combined with the pyrolysis gasoline stream 10 at a location upstream of the first stage reactor 18.

Another resultant stream 28 from the surge drum 24, which stream is preferably in a vapor phase, is routed to a depentanizer column 30, or other similar component, for removing pentane and lighter fractions from the pyrolysis gasoline stream. After processing within the depentanizer column 30, the removed C5 hydrocarbons will be in stream 32, which stream can be further processed if desired, and a vent gas stream 34 will also result. Further, the processed pyrolysis gasoline, which now lacks the C5 hydrocarbons, is routed via stream 36 to a rerun column 38 for the removal of the C9+ hydrocarbons, which exit column 38 via stream 40. Stream 40 can be further processed, as desired. As an alternative, the C9 hydrocarbons can also be removed, if desired, such that resultant stream 42 is a pyrolysis gasoline stream containing C6 to C8 hydrocarbons.

The resultant stream 42 from the rerun column 38, which in this embodiment is a pyrolysis gasoline stream containing C6 to C9 hydrocarbons (as the C5 and C9+ hydrocarbons have been removed during the fractionation process 20), is then split into a first stream 44A and a second stream 44B. Preferably, streams 44A and 44B are both liquid phase streams.

Both stream 44A and stream 44B are routed to a second stage reactor 46, which in this embodiment is preferably a hydrotreater reactor with two catalyst beds (such as an upper bed in a first portion of the reactor and a lower bed in a second portion of the reactor). In certain embodiments, the catalyst(s) and process parameters of reactor 46 are selected such that the remaining olefins and aromatics are selectively saturated, and the sulfur and nitrogen species are hydrotreated without their aromatics being saturated. The same catalyst may be used in both portions of the second stage reactor 46, or different catalysts could be used in each portion. Further, a mix of two, or more, different catalysts could be used in each portion of reactor 46, whereby either the same ratio of components of the catalyst are used in both portions of reactor 46, or different ratios of the same components are used in each of the two portions of reactor 46. Finally, it also contemplated that a reactor with more than two beds, and/or with more than two feeds, could also be used as reactor 46.

In one exemplary embodiment, the catalyst in both the first and second portions of second stage reactor 46 comprises a catalyst that is a combination of a Ni—Mo catalyst and a Co—Mo catalyst, where there is between 20-30% of the Ni—Mo component and between 70-80% of the Co—Mo component. As mentioned above, the catalyst for the first and second portions could be the same (such as a 30/70% split for Ni—Mo/Co—Mo) or two different formulations could be used (such as a 30/70% of Ni—Mo/Co—Mo for the first portion and a 20/80% split of Ni—Mo/Co—Mo for the second portion, or vice-versa).

Preferably, the second make-up hydrogen stream 16B (mentioned above) is configured to be combined with stream 44A prior to the combined stream 45 entering the second stage reactor 46. The amount of make-up hydrogen needed can be determined and controlled in any desired manner.

In the FIG. 1 embodiment, the effluent stream 48 from the second stage reactor 46 is routed to a separator 50, and the liquid phase effluent stream 52 from the separator can be split, if desired into streams 54A and 54B. Alternatively, stream 54A can be omitted because the recycle gas stream 63/66 (described below) will provide sufficient cooling for many applications.

If the optional liquid phase stream 54A is provided, it can be used as a liquid recycle feed into the first portion of the second stage reactor 46. More specifically, stream 54A, if provided, is combined with stream 44A and make-up hydrogen stream 16B to form combined stream 45, which is then directed into the first portion of the second phase reactor 46.

The stream 54B from the separator 50, via stream 52, is routed to a debutanizer 58, where it is processed to form a stream 60, which contains the C4 hydrocarbons, and a stream 62, which contains the C6 to C8 hydrocarbons. Preferably, the stream 62 is a liquid phase stream and the stream 60 is a vapor phase stream

Returning to the separator 50 of FIG. 1, in addition to the liquid phase effluent stream 52, a gas phase effluent stream 63 is also created by the separator. This gas phase effluent stream 63 is split so that it can either be routed off as vent gas via stream 64, or it can be used as recycle gas via recycle gas stream 66. As can be seen in FIG. 1, the recycle gas stream 66 passes through a recycle gas compressor 68 prior to being combines with streams 44A and 16B to form combined stream 45, which is routed into the second stage compressor 46.

Some of the advantages of the new scheme described above include the following:

(1) Hydrogen is processed Once-Through in a first stage di-olefin reactor.
(a) The current process provides for a lower hydrogen to di-olefin molar ratio than previous processes.
(b) The current process allows for a lower operating temperature than previous processes. The operating temperature ranges from approximately 40° C. to approximately 60° C. SOR (start-of-run) with a target delta temperature across the fist stage catalyst bed of 30° C. to 40° C. for embodiments of the current process. The EOR (end-of-run) temperature for the current process is usually between approximately 110° C. and approximately 120° C. In comparison, in previous processes, the SOR temperature was close to 110° C., with EOR temperatures in the range between 160° C. and 170° C.
(c) The current process provides better di-olefin (DO) saturation selectivity. The DO saturation selectivity is defined as the molar selectivity of DO to olefins saturation, and is in the range of approximately 70% to greater than 90%, dependent on the species of DO that is present in the feed to the process. This is compared to prior processes where, at the higher operating temperature, the DO to olefin selectivity ranges from between 0 to about 50%, at best, with the balance of the DO reactants proceeding all the way to the corresponding saturated paraffin or cyclic naphthenic product.
(d) The current process provides for direct liquid recycle from the first stage reactor back into the first stage reactor, without passing through the second stage reactor.
(e) In preferred embodiments of the present process, there is no recycle gas from the second stage 2nd Stage (No H₂S in Gas). The current process runs once through in terms of the feed H₂ in the first stage under very mild conditions in the liquid phase in order to achieve high DO to olefin selectivity. In addition, the fact that the first stage catalyst is fully pre-sulfided eliminates the need to recycle additional H₂ and H₂S from the second stage of the process (stream 66). This simplifies the process and allows for very high selectivity to be achieved across the first stage and maximum recycle of H₂ in the second stage where an all vapor phase condition is maintained.

(2) The make-up hydrogen can be added to both the first and the second stage reactor sections in the current process, while some previous processes only added the make-up hydrogen in the first stage reactor.

(3) The fractionation process of the current process is moved to between the first and second stages. Moving the fractionation process enables for only hydrotreating the C6 to C8 (or C9) cut, and lowers the feed rate to the second stage.

(4) The split feed to the second stage reactor eliminates the need for liquid recycle to control the delta T across the reactor (but such liquid recycle can still be provided, if desired for certain embodiments).

(5) With the current process, better aromatic retention can be expected due to controlled hydrogen addition and reduced operating severity in both the first and second stage reactors.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It is understood that various changes may be made in the function and arrangement of elements described in an exemplary embodiment without departing from the scope of the invention as set forth in the appended claims.

1. A process for treating pyrolysis gasoline comprising:
introducing a pyrolysis gasoline stream into a first stage reactor;
performing a fractionation process on the pyrolysis gasoline stream after being routed through the first stage reactor;
after performing the fractionation process, splitting the resultant stream into a first stream and a second stream; and
routing the first stream to a first portion of a second stage reactor and routing the second stream to a second portion of the second stage reactor.

2. The process according to claim 1, wherein:
the first stage reactor comprises a di-olefin reactor; and
the second stage reactor comprises a hydrotreater reactor.

3. The process according to claim 1, wherein the fractionation process comprises:
routing the pyrolysis gasoline stream through a depentanizer column; and
routing a resultant liquid stream from the depentanizer column to a rerun column.

4. The process according to claim 1, wherein the fractionation process comprises:
routing the pyrolysis gasoline stream from the first stage reactor to a surge drum;
routing a resultant stream from the surge drum to a depentanizer column; and
routing a resultant liquid stream from the depentanizer column to a rerun column.

5. The process according to claim 1, further comprising routing a hydrogen stream such that the hydrogen stream is configured to be combined with the pyrolysis gasoline stream upstream of the first stage reactor.

6. The process according to claim 1, further comprising:
routing a first hydrogen stream such that the first hydrogen stream is configured to be combined with the pyrolysis gasoline stream upstream of the first stage reactor; and
routing a second hydrogen stream such that the second hydrogen stream is configured to be combined with the second stream upstream of the second portion of the second stage reactor.

7. The process according to claim 4, further comprising combining a recycle stream routed from the surge drum with the pyrolysis gasoline stream upstream of the first stage reactor.

8. The process according to claim 1, further comprising:
routing an effluent stream from the second stage reactor to a separator; and
routing a liquid effluent stream from the separator to a debutanizer.

9. The process according claim 7 claim 8, further comprising routing a recycle liquid stream from the separator such that the recycle liquid stream is configured to be combined with the first stream upstream of the first portion of the second stage reactor.

10. A process for treating pyrolysis gasoline comprising:
routing a pyrolysis gasoline stream to a di-olefin reactor;
routing a first recycle liquid stream to the di-olefin reactor;
performing a fractionation process on the pyrolysis gasoline stream after being routed through the di-olefin reactor;
after performing the fractionation process, splitting the resultant stream into a first stream and a second stream;
routing the first stream to a first portion of a hydrotreater reactor;
routing the second stream to a second portion of the hydrotreater reactor; and
routing a second recycle liquid stream to the second portion of the hydrotreater reactor.

11. The process according to claim 10, wherein:
the first recycle liquid stream is routed from a surge drum to the di-olefin reactor; and
the second recycle liquid stream is routed from a separator to the second portion of the hydrotreater reactor.

12. A process for treating pyrolysis gasoline comprising:
routing a pyrolysis gasoline stream containing a full range of C5 to C10 hydrocarbons to a first stage reactor;
separating the C5 hydrocarbons and the C10 hydrocarbons from the pyrolysis gasoline stream after being routed through the first stage reactor; and
after separating, routing a liquid effluent stream containing C6 to C9 hydrocarbons to a second stage reactor.

13. The process according to claim 12, further comprising:
splitting the liquid effluent stream containing C6 to C9 hydrocarbons into a first stream and a second stream prior to being routed to the second stage reactor; and
routing the first stream to a first portion of the second stage reactor and routing the second stream to a second portion of the second stage reactor.

14. The process according to claim 12, wherein:
the first stage reactor comprises a di-olefin reactor; and
the second stage reactor comprises a hydrotreater reactor.

15. The process according to claim 12, further comprising:
routing an effluent stream from the second stage reactor to a separator; and
routing a liquid effluent stream from the separator to a debutanizer.

16. The process according to claim 12, further comprising;
separating the C9 hydrocarbons from the pyrolysis gasoline stream after being routed through the first stage reactor; and
after separating, routing a liquid effluent stream containing C6 to C8 hydrocarbons to a second stage reactor.

17. The process according to claim 12, further comprising routing a hydrogen stream such that the hydrogen stream is configured to be combined with the pyrolysis gasoline stream upstream of the first stage reactor.

18. The process according to claim 14, further comprising routing a hydrogen stream such that the hydrogen stream is configured to be combined with the pyrolysis gasoline stream upstream of the first stage reactor.

19. The process according to claim 12, further comprising:
routing a first hydrogen stream such that the first hydrogen stream is configured to be combined with the pyrolysis gasoline stream upstream of the first stage reactor; and
routing a second hydrogen stream such that the second hydrogen stream is configured to be combined with the second stream upstream of the second portion of the second stage reactor.

20. The process according to claim 14, further comprising:
routing a first hydrogen stream such that the first hydrogen stream is configured to be combined with the pyrolysis gasoline stream upstream of the first stage reactor; and
routing a second hydrogen stream such that the second hydrogen stream is configured to be combined with the second stream upstream of the second portion of the second stage reactor.
